(19) United States
(12) Patent Application Publication
(10) Pub. No.: US 2019/0026935 A1
(43) Pub. Date: Jan. 24, 2019

(54) METHOD AND SYSTEM FOR PROVIDING VIRTUAL REALITY EXPERIENCE BASED ON ULTRASOUND DATA

(71) Applicant: MEDIVRSE BV, Maastricht (NL)

(72) Inventors: PIOTR MICHAL PODZIEMSKI, Warsaw (PL); NITZAN MERGUEI, Maastricht (NL)

(21) Appl. No.: 15/658,257

(22) Filed: Jul. 24, 2017

(51) Int. Cl. G06T 15/08; G06F 3/01; G06T 19/20; G06T 19/00; G06F 3/0346; A61B 8/08; A61B 8/00

(52) U.S. Cl. CPC G06T 15/08; G06F 3/012; G06T 19/20; G06T 19/006; G06T 5/002; A61B 8/0866; A61B 8/4405; A61B 8/462; G06T 2219/2016; G06F 3/0346

(57) ABSTRACT

Present invention relates to a system and method for providing virtual reality (VR) experience based on fetal ultrasound data, wherein volumetric ultrasound data of the fetus previously captured on ultrasound machine is being pre-processed, at least part of the pre-processed, ultrasound data provide enough information to render representation of the fetus stereoscopically in real-time in virtual environment, the rendered representation of the fetal scan is being dynamically changed, oriented and positioned in the virtual environment based on receiving dynamically registered position and orientation of head and one or both hands of a target recipient; as well as to a computer program product and uses of the inventive method.

### BACKGROUND OF THE INVENTION

### 1. Field of the Invention

The embodiments discussed herein relate generally to a method, system and computer program product for facilitating virtual reality experience. More particularly, the embodiments discussed, herein relate to presenting to a user a virtual reality experience based on fetal ultrasound data.

### 2. Discussion of the Related Art

Today, most ultrasound machines are able to produce flat visualizations on a computer screen or machine display of the three-dimensional static (hereinafter referred to as 3D) or dynamic (hereinafter referred to as 4D) ultrasound visualizations. For example, the case of viewing an image of a baby in the womb is an important experience for the parents and should be aesthetically well made. However, when general ultrasound apparatus is used, it is sometimes difficult for a patient to recognize a part being shown in an ultrasound image. Especially, currently used visualizations may fail to provide feeling of meeting between parents and their soon to be born baby, because visualized ultrasound images appear very clinical on the computer screen and the interaction between the scan and parents is usually confined to viewing the image representing their baby. Recognition of the features of the baby may seem difficult and inconvenient.

### SUMMARY OF THE INVENTION

Several embodiments of the invention advantageously address the needs above as well as other needs by providing a method,, system and computer program product for facilitating virtual reality experience. More particularly, the embodiments discussed herein relate to presenting to a user a. virtual reality experience based, on fetal ultrasound data.

In one embodiment, the invention can be characterized as a method for providing an interactive virtual reality experience of a virtual representation of fetus to one or more users, the representation of fetus being provided based on static 3D and/or dynamic 4D volumetric data of one or more fetal ultrasound scans, wherein said volumetric data represent acoustic echoes from the fetal and maternal tissues, the method comprising obtaining static 3D and/or dynamic 4D volumetric data of one or more fetal ultrasound scans, wherein said volumetric data is obtained responsive to a file import of a file associated with the ultrasound machine software; determining virtual reality information representing a virtual environment, wherein at least part of the said environment is based on said volumetric data, comprising: receiving an. input containing information of a location and rotation of a head of the user in the real-world physical space; receiving an input containing information of a location and orientation of one or more hands of the user in the real-world physical space; calculating at least one of the following: new position, scale and/or orientation of the representation of the fetal scan in the virtual reality environment, wherein the new position, scale and orientation is responsive to received input of the location and rotation of the head, of the user and the received input of the location and the orientation of one or more hands of the user; and rendering the representation of one or more fetal scans for each eye of the user through volume rendering methods applied to the said volumetric data, in the calculated position and orientation; displaying the determined virtual reality information using a near-eye display system for providing the interactive virtual reality experience.

This Summary is provided to introduce a selection of important concepts in a simplified form that are further described below in the Detailed Description of Example Embodiments. This Summary is not intended to be used to limit the scope of the present disclosure. This Summary is not intended to identify key features of the claimed subject matter.

The details of one or more implementations are set forth in the accompanying drawings and the description below. These, additional and/or other features of the embodiments of the present invention will be inferable from the description and drawings, and from the claims, or can be learned by the practice of the embodiments set forth herein.

### BRIEF DESCRIPTION OF THE DRAWINGS

The above and other aspects, features and advantages of several embodiments of the present invention will be more apparent from the following more particular description thereof, presented in conjunction with the following drawings.

In the accompanying figures:

FIG. 1 illustrates one example of an implementation of a near-eye display system for providing virtual reality content to a user according to the prior art.

FIG. 2 illustrates one example of a system configured for providing a user virtual reality experience based on fetal ultrasound data according to some embodiments of the present invention.

FIG. 3 illustrates another example of a system configured for providing a user virtual reality experience based on fetal ultrasound data according to some embodiments of the present invention.

FIG. 4 illustrates one example of a near-eye display system depicted in FIG. 2 according to some embodiments of the present invention.

FIG. 5 is a flowchart diagram illustrating a possible realization of a method of providing virtual reality experience based on fetal ultrasound data to a user, in accordance with some embodiments of present invention.

FIG. 6 illustrates one example of a method of providing VR experience based on fetal ultrasound data in action, in accordance with some embodiments of the present invention.

FIGS. 7A and 7B shows one example implementation of performing interaction with an ultrasound scan representation in virtual reality environment, in accordance with some example embodiments of the present invention.

FIG. 8 shows one example of an implementation of an interface that facilitates a user to import data associated with fetal ultrasound scan and prepare virtual reality experience, according to some embodiments of the present invention.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION

The following description is not to be taken in a limiting sense, but is made merely for the purpose of describing the general principles of exemplary embodiments. The scope of the invention should be determined with reference to the claims.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

As used herein, references to the “present invention” or “invention” relate to exemplary embodiments and not necessarily to every embodiment encompassed by the appended claims.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

A better recognition of 3D features embodied in ultrasound data may come from virtual reality systems. A virtual reality (VR) system may generate a three-dimensional (3D) stereoscopic, immersive virtual environment. As used herein, references to the “virtual reality” relate to both virtual reality in a narrow sense of completely artificially generated virtual environment, and to augmented reality (AR)—a form of VR that layers virtual environment elements over real surrounding around the user. A user or targeted recipient may experience this virtual reality environment by viewing computer generated virtual information including two-dimensional virtual objects and/or three-dimensional virtual objects. Such objects are commonly rendered based on 3d polygon meshes or collections of vertices, and not based on volumetric representation. User may also interact with virtual environment through means of various electronic devices, including but not limited to a head mounted device or glasses including a display, gloves or hand-held controllers fitted with sensors or recognizable markers, depth cameras mounted on the head mounted device or near the user and other such electronic devices. In the virtual reality system, a user interacts with visual information generated by software to experience a new location, activity, etc.

However, the development of an immersive paradigm that provides for user engagement with ultrasound data via virtual reality devices has proven elusive, as the widely used approaches and methods for creating VR content are not straightforwardly conductive to visualization of ultrasound data. Thus, there remains a considerable need for systems and methods that can conveniently visualize ultrasound data in virtual reality. Moreover, the way of interaction with the visualization of ultrasound data, may be paramount for the user engagement into such virtual reality experience.

The term “Virtual Reality” (VR) as used herein is defined as an artificially generated virtual environment that can provide an illusion of being present in the real or imagined space. Virtual reality can recreate sensory experiences, such sight, sound, touch and similar. Traditional VR systems apply near-eye displays for presenting the environment to user to simulate 3d vision.

The term “Augmented Reality”(AR) as used herein is defined as a form of VR that layers virtual environment elements over real surrounding around the user. Traditionally, this can be done either by adding computer-generated input to a live direct view of real world by using semi-transparent displays, or by layering virtual information over a live camera into near-eye displays.

The term ‘near-eye display’ as used herein is defined as a device, including one or more displays, usually wearable on the head. The displays usually provide stereoscopic visual information—each eye is presented with a slightly shifted representation of environment. The device may include optical systems to adjust the provided visual information to the eye. The device also includes means for holding the display in the form of googles, headset or similar. The term ‘near-eye display’ will be used interchangeably with the terms ‘virtual reality headset’, ‘googles’ or ‘head mounted display’.

The term ‘near-eye display system’ as used herein is defined as a device, comprising the near-eye display, together with processing hardware able to prepare virtual reality information to be provided to a user, and/or other components.

A “virtual environment”, also referred to as a “virtual scene”, “virtual reality environment” or “virtual surrounding” denotes a simulated (e.g., programmatically), spatially extended location, usually including set of one or more objects or visual points of reference, that can give a user a sense of presence in a surrounding different than his actual physical environment. The virtual environment is usually provided to a user through a near-eye display. It may take over a user's field of view partially or completely, and give the user a sense of presence inside a virtual reality experience.

Various embodiments of the present disclosure can include methods, computer program, non-transitory computer readable media and systems for facilitating virtual reality experience based on fetal ultrasound data.

In one aspect, a method may include providing an interactive virtual reality experience of a virtual representation of fetus to one or more users, the representation of fetus being provided based on static 3D and/or dynamic 4D volumetric data of one or more fetal ultrasound scans, wherein said volumetric data represent acoustic echoes from the fetal and maternal tissues. The method may include the following steps: obtaining static 3D and/or dynamic 4D volumetric data of one or more fetal ultrasound scans, wherein said volumetric data is obtained responsive to a file import of a file associated with the ultrasound machine software; determining virtual reality information representing a virtual environment, wherein at least part of the said environment is based on said volumetric data; and displaying the determined virtual reality information using a near-eye display system for providing the interactive virtual reality experience.

To achieve this determining virtual reality information representing a virtual environment may include the following steps: receiving an input containing information of a location and/or rotation of a head of the user in the real-world physical space; receiving an input containing information of a location and orientation in the real-world physical space of one or more hands of the user; calculating at least one of the following: new position, scale and/or orientation of the representation of the fetal scan in the virtual reality environment, wherein the new position, scale and orientation is responsive to the received input of the positions of head and/or one or more hands of the user in the physical real-world; and rendering the representation of one or more fetal scans for each eye of the user through volume rendering methods applied to the said volumetric data, in the calculated position and orientation.

In another aspect, in accordance with one embodiment, a system implementing the method for providing an interactive virtual reality experience of a virtual representation of fetus to one or more users may include a near-eye display configured to project a synthetic virtual scene, into both eyes of a user, so as to provide a virtual reality view to the user; means of determining position of hands in a real physical-space; a memory storing executable machine-readable instructions; and computational processing hardware containing one or more physical processors configured by machine readable instructions capable of performing the method for providing an interactive virtual reality experience of a virtual representation of fetus to one or more users.

In another aspect, a non-transitory computer readable medium containing program instructions for causing a computer to perform the method for providing an interactive virtual reality experience of a virtual representation of fetus to one or more users is provided herein.

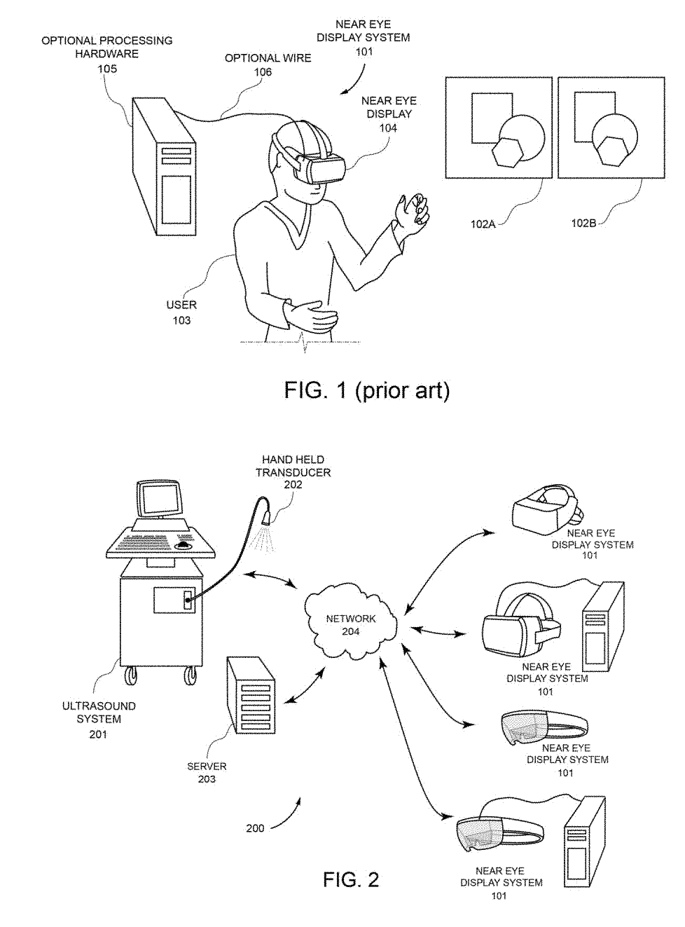

FIG. 1 shows a traditional VR system, in which an example near-eye display system 101 is projecting a computer generated virtual image 102A and 102B onto each eye of the user 103 through a near-eye display 104. The virtual images 102A and 102B are stereoscopic in the sense, that each eye receives the visualized information at a slightly different angle, to simulate human 3d vision. Usually, near-eye display 104 includes sensors such as accelerometers or gyroscopic sensors (not shown), that can detect in real time viewing angle of the user 103, to adjust presented virtual images 102A and 102B. This adjustment creates the sense of presence—providing an illusion of a stable virtual environment. Additionally, near-eye display system in some implementations can include external processing hardware 105 (e.g. a PC, smartphone, laptop, other graphics hardware) coupled to a near-eve display system such that the near-eye display system can actually consist of multiple discrete, connected hardware components, wherein connections may be done for example via cable wire 106 or wireless. Common examples of such implementations are the Oculus Rift™ system, HTC Vive™, Metavision Meta™ 2 glasses and similar. In some implementations, near-eye display system contains the processing hardware within the headset, without externally visible processing hardware. Common examples of such implementations are Gear VR™ headset or Hololens™ headset.

The subject matter claimed herein is not limited to embodiments that solve any disadvantages or that operate only in environments such as those described above. Rather, this background is only provided to illustrate an example technology area where some embodiments described herein may be practiced.

Various embodiments of the disclosure are discussed in detail below. While specific implementations are discussed, it should be understood, that this is done for illustration purposes only. A person skilled in the relevant art will recognize that the claimed subject matter may also be embodied in other ways, and other components and configurations may be used without parting from the spirit and scope of the disclosure. The phraseology and terminology used hereinafter is for the purpose of description and is not intended to limit the scope of the present invention. Moreover, although the terms “step”, “part”, “operation” and/or “block” may be used in the following description to describe different elements of methods employed, the terms should not be interpreted as implying any fixed order of various steps of the method disclosed herein, unless when the order of particular steps is explicitly denoted to be necessary.

Some embodiments of the present invention relate to a method and system for providing virtual reality (VR) experience based on fetal ultrasound data. For example, a user immersed in a virtual reality environment wearing, for example, a near-eye display such as a head mounted display, may view and interact with the representation of the fetus based on ultrasound data captured by an ultrasound machine. In some embodiments, the virtual reality (VR) experience can be provided after the visit for the ultrasound scan has finished, for example in separate room or even much later, at a remote location (e.g. at home of the parents).

In some embodiments of the present invention, a user may be enabled to import the volumetric ultrasound data of the fetus previously captured by an ultrasound machine by a file import component, or any other means capable of obtaining the ultrasound scan data. Based on imported volumetric ultrasound data of the fetus, the virtual representation of the fetus is being prepared. Said step of preparation of virtual representation will be also generally referenced hereinafter as “pre-processing step” and a component configured to facilitate “pre-processing step” will be referred to as the “pre-processing component”. In some embodiments of the present invention, during the pre-processing step imported volumetric ultrasound data may be altered, a piece of data may be removed and/or some data may be generated to provide a better visual representation of the fetus. In some embodiments, the prepared representation of the fetus may consist only of representation of a part of the fetal body. i.e. head, face, part of the face, torso, hand and/or any other part of the body and/or the fetus.

Some embodiments of the present invention render prepared representation of the fetal scan in the virtual environment for the user, for example using volume rendering methods, allowing him or her to interact with the scan.

More specifically, in some embodiments of the present invention a user may move his or her body parts, for example, head or one or more hands, in a manner that is translated in real time by embodiments of the present invention to move, rotate, and position the representation of the fetal scan. For example, to a user such movements may provide a feeling of holding the representation of fetus in one or more hands, touching it, caressing the skin and other. In another example, such movements may provide a feeling that the representation of the fetus is floating in the vicinity of the hands, and is responding to a movement of the user's body parts with some delay.

Referring to the drawings in general, to improve readability of the description, any user that could be viewing a virtual reality environment through a near-eye display system will be referenced as user 103.

FIG. 2 illustrates a system 200 for providing user 103 a virtual reality experience based on fetal ultrasound data in accordance with some embodiments of the present invention. In some implementations, as shown in this example, system 200 may include an ultrasound machine 201 able to perform an ultrasound scan through an ultrasound transducer 202. In some implementations, as shown in this example, system 200 may also include a server 203.

In some embodiments of the present invention, the ultrasound machine 201 may be of a cart type apparatus or portable type apparatus. Examples of portable ultrasound machines, may include, but are not limited to a picture archiving and communication system (PACS) viewer, tablet, mobile phone, and a laptop computer.

The ultrasound machine 201, the near-eye display system 101, and/or server 203 may be operatively linked via one or more means of communication and/or transfer of data, that may be a wireless network 204 (for example WiFi or Bluetooth among others), a cable network connection (not shown), an external storage memory (not shown), or other means of exchange of data and information between elements of the system.

Linked elements of the system 200 comprising of the ultrasound machine 201, the near-eye display system 101, and/or server 203 may operate together, or separately, to facilitate various steps of the method of delivering virtual reality experience based on ultrasound data as will be described hereinafter in various embodiments of the present invention.

The near-eye display system 101 may include one or more processors configured to execute computer program components. The computer program components in some embodiments may be configured to facilitate a user to import the ultrasound scan, pre-process the ultrasound data to produce a virtual representation of the fetus, provide to the user 103 a virtual reality experience based on virtual representation of the fetus, enable the user 103 to interact with the virtual environment and/or provide another functionality.

Some examples of the near-eye display system 101 are illustrated in FIG. 2, which, as shown, may include a head-mounted device, augmented reality glasses, a head mounted device coupled with external processing hardware (e.g. PC, smartphone or other graphics hardware), augmented reality glasses coupled with external processing hardware, and/or any other types of near-eye display systems that can provide a virtual reality information to a user.

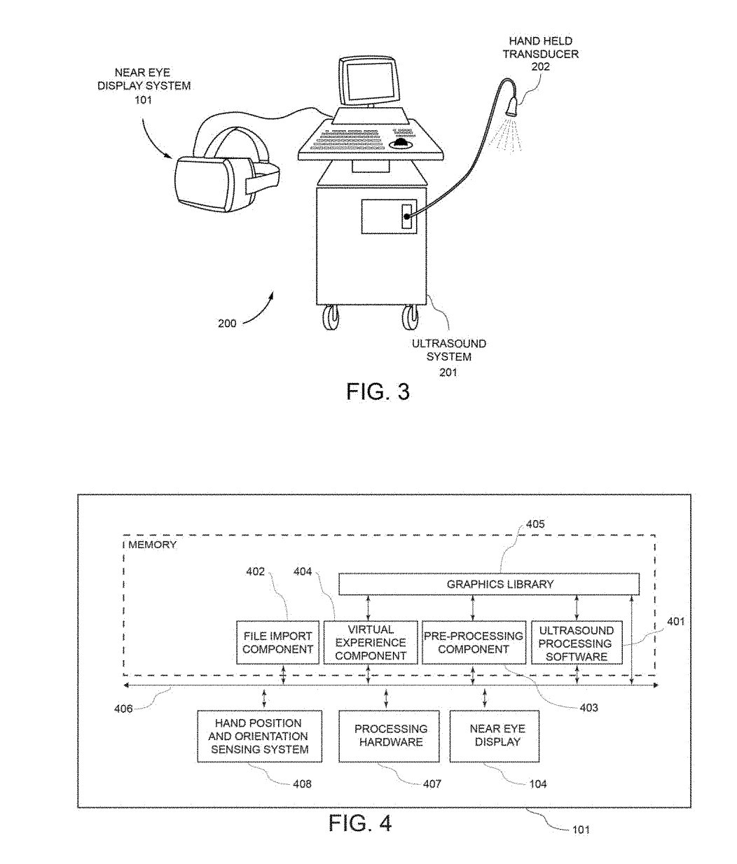

In another embodiment, as shown in FIG. 3, the system 200 may be constructed in a way, that near-eye display system 101 is coupled with ultrasound machine 201 directly via a cable or cable-less connection, thereby, at least partially, the ultrasound machine 201 may serve the purpose of the external processing hardware 105 and perform at least some of the functional steps of the method for providing a user a virtual reality experience based on ultrasound scan data of a fetus. For example, rendering of the visual information presented to each eye of the user in this embodiment may be performed on the ultrasound machine 201.

FIG. 4 illustrates one example of near-eye display system 101 illustrated in FIG. 2. As shown, the near-eye display system 101 may include ultrasound processing software 401, file import component 402, pre-processing component 403, virtual experience component 404 (referred to also as “virtual reality application”), graphics library 405, a system bus 406, processing hardware 407, a system for sensing hand position and orientation 408, a near-eye display 104 and/or any other component.

It should be understood that the components shown in FIG. 4 included in the near-eye display system 101 are merely illustrative of one example embodiment and this configuration is not intended to be limiting, rather it is presented to show one of possible configurations. In various implementations near-eye display system 101 may include less or more components than those shown in FIG. 4.

In one example implementation (described with reference to FIG. 2 and FIG. 4), where the system 200 includes both server 203 and the ultrasound machine 201 next to the near-eye display system 101, the near-eye display system 101 may not include components such as ultrasound processing software 401, file import component 402 or pre-processing component 403. Instead, the ultrasound processing software 401 may be located in the ultrasound machine 201, and file import component 402 and pre-processing component 403 may be located in the server 203. In such example configuration, the ultrasound data of the fetus may be imported from the ultrasound machine 201 to the server 203, where the pre-processing component 403 prepares virtual representation of the fetus, which is subsequently transferred to the near-eye display system 101, where the virtual environment is constructed using virtual experience component 404 and presented to a user.

In another example configuration, the ultrasound processing software 401 may be located in the ultrasound machine 201, and components 402 and 403 may be located in the near-eye display system 101. In such example configuration, the ultrasound data of the fetus may be exported from ultrasound processing software 401 on the ultrasound machine 201 to the external storage memory (not shown), which can be then coupled to the near-eye display system 101.

Returning to FIG. 4, the file import component 402 may enable a user to import one or more files associated with one or more ultrasound scan data from any of following storage locations in the system 200: local memory in the near-eye display system, memory of the ultrasound machine 201, server 203, an external storage memory and/or any other storage location. The file associated with ultrasound scan data may include various formats. Some examples of proper file format extensions may include .DCM, .RAW, .VOL, .TIFF, .PNG, .JPG, .NII, .IMG and/or any other file extensions.

The pre-processing component 403 may be configured to facilitate preparation of the virtual representation of the fetus in the pre-processing step, including but not limited to filtering out the structural noise present in the said volumetric data of fetal ultrasound scan using at least one or more of filtering methods executed by processing hardware 407; adjusting the visual parameters of said virtual representation of the fetus, (e.g. parameters such as opacity, color brightness) and/or adjusting parameters of volume rendering methods applied to the said volumetric data (thus setting configuration of the virtual experience component 404). The pre-processing step will be described in more detail hereinafter.

The virtual experience component 404 can be configured to determine virtual reality information representing a virtual environment. The determination can include but is not limited to providing a view of the virtual environment and/or other information that describe the virtual environment to user 103, rendering the representation of one or more fetal scans for each eye of the user 103, and/or calculating new positions, scale and orientations of the representation, of the fetal scan in the virtual reality environment. In some embodiments, the experience component 404 may also provide additional content (e.g. text, audio, pre-recorded video content, pre-recorded audio content and/or other content) as a part of the virtual environment presented to the user 103. The file import component 402, pre-processing component 403, and virtual experience component 404 may communicate with a graphics library 405. The graphics library 405 may provide a set of graphics functionalities by employing graphics hardware, which may be a part of the processing hardware 407. For example, the near-eye display system 101 may include processing hardware 407 comprising of one or more Computational Processing Unit (CPU) processors and/or graphics hardware including Graphical Processing unit (GPU). The processing hardware 407 may be configured to distribute a computational workload of preparing virtual reality experience by components 402, 403, and 404 between the CPU and the GPU with help of the graphics library 405. Common examples of the graphics library 405 may include DirectX, OpenGL or any other graphics library. In one implementation, without limitation, the virtual experience component 404 may be configured with a Unity® game engine, Unreal® Engine game engine, or Cryengine™ game engine.

The near-eye display system 101 may also include or may be in operative association with a hand position and orientation sensing system 408, which will be described hereinafter.

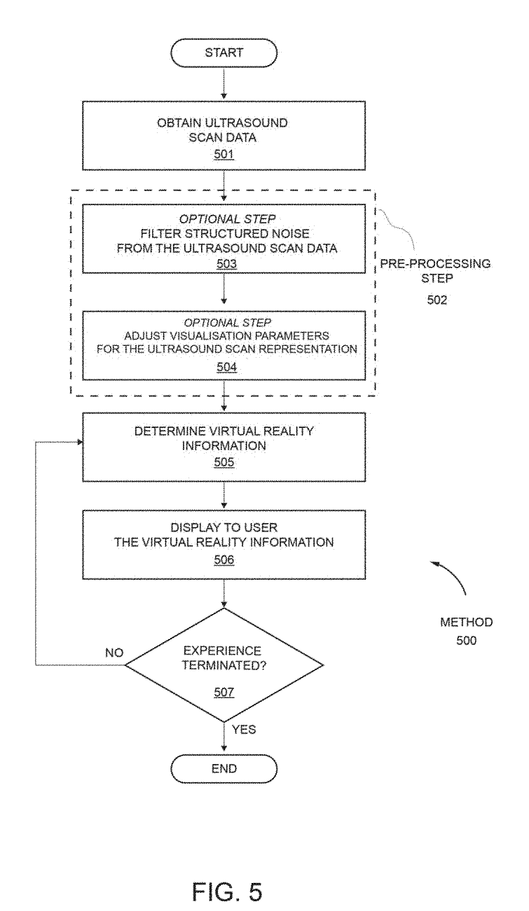

FIG. 5 is a flowchart diagram illustrating a method 500 in accordance with some embodiments of the present invention. Note, that the method 500 may be accomplished with one or more additional operations not described herein, and/or without one or more of the operations discussed. In addition, the logic flow of the method 500 depicted in FIG. 5 do not require the sequential order shown, and the illustrated order of the operations is not intended to be limiting. The process starts off with the operation 501 in which ultrasound scan data is being obtained. The information obtained may include: one or more static (3D) or dynamic (4D) ultrasound scan data sets forming volumetric data representing acoustic echoes from the fetal and maternal tissues; physical measures such as dimensions of the volumetric data sets, spacing between measurement points of acoustic echoes, estimated size of the fetus, and/or any other information. In some implementations, the operation 501 may be performed by the same or similar component to the file import component 402 shown in FIG. 4.

In some embodiments, at an operation 502, referred to before as a pre-processing or pre-processing step, preparation of the virtual representation of the fetus may be conducted, including but not limited to filtering out the structural noise present in the said volumetric data (sub-step 503); and/or adjusting parameters of volume rendering methods (sub-step 504) applied to the said volumetric data in the following step 505.

In some embodiments, operation 502 may also include a step allowing a user to remove at least part of the volumetric data of the ultrasound scans, that is deemed not relevant by the user. Such information may be in a non-limiting example a piece of data representing maternal tissue in an abdominal scan of a fetus. This may be advantageous to the virtual experience provided to the user 103, as, for example, the view on important parts of the representation of the fetus (e.g. face) would not be occluded by not relevant parts of the ultrasound scan data, like parts of maternal body.

In some embodiments, step 502 may also include a step, in which the volumetric data is divided into subgroups, using various classifying algorithms, for storing in various data structures that may differ from the original data structure. Some non-limiting examples of said structures may be octrees, look-up tables, voxel octrees, billboard tables, summed area tables, summed volume tables and/or any other.

In some embodiments, operation 502 may also include steps, in which various elements of the virtual reality environment are prepared, including one or more points of references such as floor, sky, walls, or any other virtual objects, that, for example, may enhance and improve the feeling of immersion for the user 103. Said various elements may further include light and lighting, textures, floating particles, shadows, lightings and other visual information.

In some embodiments, said filtering of the volumetric data during sub-step 503 may be performed to filter the structural noise present in the ultrasound data, wherein the structural noise may comprise of the speckle noise, directional decrease of signal attenuation and/or any other type of unwanted distortion of ultrasound scan data. The filtering may be performed using one or a combination of various filtering and image processing algorithms, for example, executed by processing hardware 407 with workload distributed between CPU and GPU. The combinations may include filtering methods known in literature like median filtering or average filtering, more sophisticated methods both known in art or original or any other processing method.

After steps 501 and 502 are completed, the interactive virtual experience may start for user 103, provided by operations 505 and 506 executed in a looping manner until it is determined that the virtual reality experience has been terminated (block 508). The loop marks the part of the method, during which the user 103 may be immersed in the virtual reality experience. In some embodiments, both operations 505 and 506 may be performed by the component similar or the same to the virtual experience component 404.

At operation 505, virtual reality information representing a virtual environment may be determined. According to a possible implementation, the process may receive an input containing information of a location and/or rotation of a head of user 103 in the real-world physical space (for example from sensors in the near-eye display 104). Furthermore, the process may also receive an input containing information of a location and orientation of one or more hands of the user 103 in real three-dimensional space, received from hand position and orientation sensing system 408.

Once all the inputs are collected, a position, scale and orientation of the representation of the fetal scan in the virtual reality environment may be calculated, wherein the new position and orientation is responsive to received input containing information of position and orientation of at least one of the following: head and one or more hands of the user 103. It is to be noted, that the new orientation of the representation of the fetus in relation to the user point of view in virtual reality, may be different than the originally registered orientation of the ultrasound scan. An example description of calculating the position, scale and orientation of the representation of the fetal scan will be detailed hereinafter.

In some embodiments, based on the position, scale and orientation of the representation of the fetus in relation to the position and rotation of the head of the user 103, the visual properties of the fetus may be adjusted during operation 505, by changing parameters of the representation of the fetus, where said parameters are from a group of opacity, color and brightness. For example, this may allow to make the representation of the fetus transparent, if the current orientation of said representation shows to the user 103 a site corresponding to a very noisy part of the ultrasound scan.

Once the position, scale and. orientation of the representation of the fetal scan is calculated, rendering of the representation may be performed, for example by using various volume rendering methods. The volume rendering methods for example may comprise of ray casting algorithm. In a ray casting algorithm, computational rays are emitted from the position of both eyes of the user 103 through each sample of the virtual representation of the fetus, located and placed in the virtual environment. Each computational ray passes through the representation of the fetus, which is containing volumetric ultrasound data, and re-samples the volume data, producing a value of a pixel color and opacity synthesized according to a mathematical model of the optical properties of the light propagation model within the tissue represented by ultrasound data, wherein the calculated pixel corresponds to physical pixel on the near-eye display 104. The parameters of the applied volume rendering methods may be set up during operation 503, prior to delivering a virtual reality experience to the user.

In some embodiments, the ray casting algorithm may be performed in a following, non-standard way. First, positions of fragments on the front facing part of the bounding box of the scan volume are rendered to a texture, without writing to the depth buffer. In the next step, positions of the fragments on internal back facing part of the bounding box are rendered to a texture, with writing the content to the depth buffer. Such reversed order of rendering front-facing part of the bounding box, then back facing part allows to render the representation of the fetus positioned on the internal back faces of the bounding box, allowing for placing the point of view (i.e. eye positions of the user 103) inside the bounding box. This prevent appearing of artifacts due to the clipping the data visualized on the front faces by camera near-eye plane, that can happen in a standard ray-casting volume rendering approaches. This method also prevents the near-eye plane to clip the volume bounding box without significant GPU computational overhead as it does not require to calculate intersection between the volume bounding box and the camera near-eye plane. Then, computational rays are emitted from the position of both eyes of the user 103 towards the back faces of the internal part of the volume bounding box of the representation of the fetus. The computational rays travel through each sample of the virtual representation of the fetus, located and placed in the virtual environment. Each computational ray passes through the representation of the fetus, which is containing volumetric ultrasound data, and re-samples the volume data, producing a value of a pixel color and opacity synthesized according to a mathematical model of the optical properties of the light propagation model within the tissue represented by ultrasound data, wherein the calculated pixel corresponds to physical pixel on the near-eye display 104.

In some embodiments, in the generated virtual environment other elements may be rendered, including for example one or more points of references prepared in operation 502 such as floor, sky, walls, visual representations corresponding to the actual position and orientation of at least one hand of the user in the virtual reality environment based on the received position and orientation of at least one hand of the user in the real-world physical space, and/or any other virtual objects.

In some embodiments, the generated virtual environment may be augmented by other elements, such as pre-recorded audio content, including but not limited to musical ambient background, narration audio, fetal heart beat.

At operation 506 all the determined and rendered virtual information during operation 505 may be displayed through the near-eye display system 104.

Note that operations 501-504, in some embodiments of the present invention may be assisted by a second user different from user 103, whenever a user action may be necessary (for example deciding which ultrasound data scan should be imported or during deciding on any of the parameters and configurations during steps 501-504.

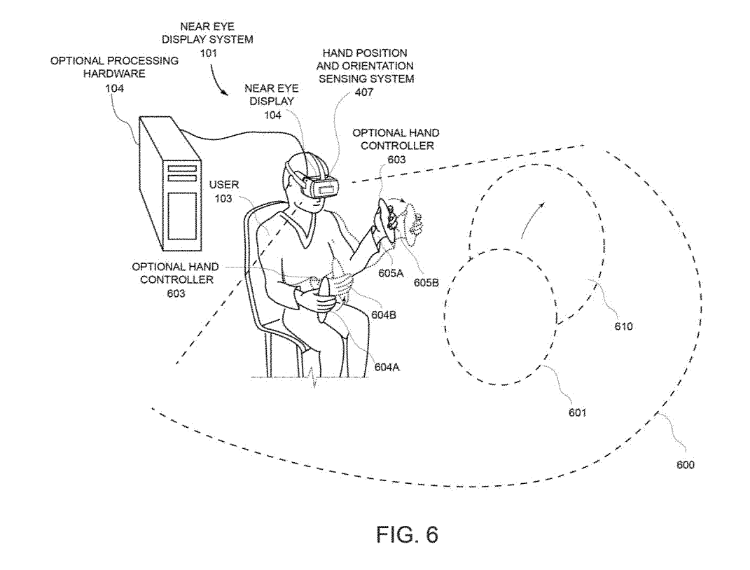

FIG. 6 is a schematic diagram illustrating a system 200 performing a method 500 in action, according to one embodiment of the present invention. FIG. 6 is a third-person view of the user 103, including a view of virtual environment 600 generated by the near-eye display system 101, wherein the virtual environment is including but is not limited to a view of the representation of the fetus 601. Note, that the shown virtual environment 600 with representation of the fetus 601 would be viewed by the user through the near-eye display system 101, and the depiction of the virtual environment 600 outside of the near-eye display 104 is simply for ease of explanation and illustration. Note also, that depicted position and scale of the representation of the fetus 601 may not reflect the real position and scale as perceived by the user 103, and rather it was used for clarity of the illustration.

As shown in FIG. 6 a user immersed in virtual reality environment 600 may explore and engage in interaction with any object being a part of the environment 600, including but not limited to the representation of the fetus 601. User 103 may be able to freely look into any direction of the virtual environment 600 through near-eye display 104. Ability to decide to look into certain direction and interact with the environment 600 may provide a feeling of personalization of the experience for the user 103. In one non-limiting configuration, interaction may be exercised through control input by the user 103 through hand position and orientation sensing system 407. In another non-limiting configuration interaction may be exercised through control input by the user through hand controllers 603 held in one or both hands.

The hand position and orientation sensing system 407 (referred to also as hand location device) may include one or more cameras, such as an infra-red camera, an image sensor, an illuminator, such as infrared light source, a light sensor, to capture moving images, that may be used to help track a physical position and orientation of user's 103 hands and/or hand controllers 603 in the real world. In operation, device 407 may serve as a means for determining position of hands. The device 407 may be a commercially available hardware such as LeapMotion™ or RealSense™ or any other similar hardware. In another example embodiment, hand controllers 603 may serve in operation as a means for determining position of hands. The hand controllers 63 may be a commercially available hardware such as Oculus Touch™ or HTC Vive™ controllers or any other similar hardware.

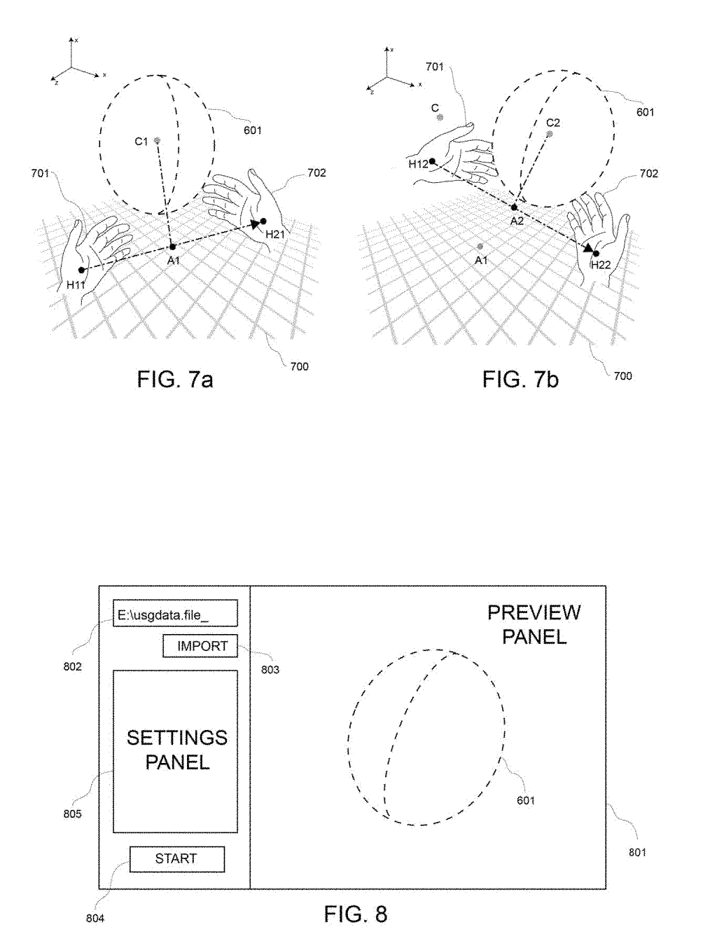

As shown in FIG. 6, according to one embodiment of the present invention, user 103 by movement of hands from positions 604A and 605A to positions 604B and 605B is able to apply a new rotation, position and scale to the representation of the fetus 601, wherein the representation of the fetus in the new rotation, position and scale is depicted schematically as element 610. FIG. 7a and FIG. 7b illustrate one example of how the new rotation, position and scale of the representation of the fetus 601 may be determined responsive to received input from hand location device 407 containing information of the position and orientation, of one or both hands of the user.

Figures FIG. 7a. and FIG. 7b illustrate an example of what user 103 may view through the near-eye display. The grid 700 schematically represents the virtual reality environment elements.

In one non-limiting example, once the movement of one or more hands of user 103 is being detected, metrics of the movement in the real world such as direction and displacement of one or more hand are translated into the movement of one or more representations of user's hand 103. FIG. 7a illustrates an example of an initial situation in the virtual reality environment, wherein H11 marks exemplary initial position of the representation of the first hand 701 of user 103, H21 marks exemplary initial position of the representation of the second hand 702 of user 103, C1 marks exemplary position of the representation of the fetus. Note, that the virtual representations of the hands (701 and 702) in some embodiments may not necessarily be in a shape of hands, instead it may be represented by, for example, a sphere, point, circle or any other shape or rendering. The shape of hands In the FIG. 7a and FIG. 7b is for the purpose of ease of description. FIG. 7b illustrates an example of situation, after user 103 moved his/her hands. H12 and H22 marks moved position and orientation of the representations of respectively first (701) and second (702) hand of user 103. Once the movement of one or more hands is performed, metrics associated with their respective positions are being further analyzed in order to translate said metrics into movements and scaling of the representation of the fetus 601. Some examples of such metrics may be: a weighted average of the positions of both hands of the user (marked as A1 in initial situation and as A2 when hands are moved as shown in FIG. 7a and FIG. 7b); the direction and magnitude of the vector between the positions of the representations of the first and second hand, of the user (vector between points H11 and H21 in initial situation and between points H12 and H22 when hands are moved as shown in FIG. 7a and FIG. 7b or position and displacement of one hand only.

In some embodiments of present invention, the translation of said metrics into movements and scaling of the representation of the fetus 601 in the virtual reality environment may be realized through applying a velocity and/or acceleration to the representation of the fetus 601, proportional to changes in said metrics. Applied velocity and/or acceleration may cause the representation of the fetus 601 to scale, rotate, and move from the position C1 to the position C2 as shown in FIG. 7a and FIG. 7b, when user 103 moves his or her hands. Such a way of translating the movement of hands into the movement, rotation and scaling of representation of the fetus 601 allows the movement, rotation and scaling of representation of the fetus 601 to continue for some time after user 103 stopped to move hands. Such continuing movement may provide a feeling that the representation of the fetus floats in the vicinity of hands, and responds to a movement of user body parts with some delay.

FIG. 8 illustrates a non-limiting example of an interface 801, that facilitates a user to import one or more files associated with one or more fetal ultrasound scans, initialize and set up the pre-processing step, and start the virtual reality experience for a user. The generation of the interface 801 may by implemented on a near-eye display system 101, for example, as part of a component similar or the same as the file import component 402. In another embodiment, the generation of the interface 801 might be implemented on a server 203 as a web interface and displayed on a near-eye display system provided through a standard web browser. Interface 801 may be for example displayed on a standard monitor (for example connected to the processing hardware 105) or directly on the near-eye display of a near-eye display system. As shown in FIG. 8, the interface 801 may comprise of field controls 802,803,804 and any other field controls. The field control 802 (an input box for example) may be provided to enable a user to select file(s) containing the ultrasound scan data (e.g. with file extension as described before). The file(s) may reside for example on the near-eye display system 101 or on an external storage memory connected to the near-eye display system 101. In various embodiments, the file(s) may reside on the server 203 or on the ultrasound machine 202, with which the near-eye display system may be connected through the network 204 or other means of communication. The field control 803 (e.g. a button) may be provided to enable a user to load the ultrasound scan data from the file to the pre-processing component 403. The field control 804 (e.g. a button) may be provided to enable a user to start the virtual reality experience for the user, or for another user wearing a near-eye display of the near-eye display system. As shown in FIG. 8, in some implementations, the interface 801 may further comprise of settings panel 804, which may be provided to enable a user to set up one or more parameters of the method 500, for example during step 502. In some embodiments the settings panel 804 may further comprise of a field control (not shown) that may be provided to enable a user to select a path (including but not limited to a local path on the near-eye display system 101, or a path on the server 203) to save a file after providing a virtual experience to the user, wherein said file may contain original ultrasound data, prepared representation of the fetus after the pre-processing step 502, parameters of the method 500 set up by the user, measures of user's body parts movement, including but not limited to head and one or more hands, and any other information. As shown in FIG. 8, in some implementations, the interface 801 may further comprise a preview of the virtual representation of the fetus 601, provided to enable a user to preview the representation of the fetus before starting the virtual experience.

Toggling the representation of the fetus: According to some embodiments, the hand position and orientation sensing system 407 may enable the user to perform a gesture or manual operation that will result in the change between different representations of the fetus prepared during the pre-processing step 502. For example, an interface in form of an element of the virtual environment (e.g. 3d button, switch) may be provided to a user 103 to enable user 103 to change the representation of the fetus by clicking or grabbing the interface element. In another embodiment, the user 103 may perform a gesture (e.g. horizontal movement of one of his hands) that will be interpreted by the system 200 and result in changing the representation of the fetus.

Multiple users: According to some embodiments, the system 200 may include more than one near-eye display, thereby enabling the system to provide the virtual reality information to plurality of display devices associated with to plurality of users. In such embodiment, the method 500 would enable at least two users to interact with the virtual reality environment via their respective display devices substantially simultaneously.

Although various features of the present invention may be described herein in the context of one embodiment, it does not preclude that these features may be also implemented separately or in any configuration suitable for realization of the present invention. Moreover, features of the invention described herein in the context of separate embodiments may also be realized in a single embodiment of the present invention.

It is to be understood that where the claims or description of example embodiments refer to “the”, “a” or “an” element, it does include plural referents unless it is clearly apparent otherwise from the context.

It is to be understood that the methods of the present invention may be implemented by performing selected operation manually, automatically or in any combined way.

It is to be understood, that the description of only a limited number of embodiments presented here should not be treated as limitation of the scope of the invention, but rather as examples of some of the preferred implementations. Other possible changes, variations and applications may be also within the scope of invention.

While the invention herein disclosed has been described by means of specific embodiments, examples and applications thereof, numerous modifications and variations could be made thereto by those skilled in the art without departing from the scope of the invention set forth in the claims.

What is claimed is:

1. A method for providing an interactive virtual reality experience of a virtual representation of fetus to one or more users, the representation of fetus being provided based on static 3D and/or dynamic 4D volumetric data of one or more fetal ultrasound scans, wherein said volumetric data represent acoustic echoes from the fetal and maternal tissues, the method comprising:
a obtaining static 3D and/or dynamic 4D volumetric data of one or more fetal ultrasound scans, wherein said volumetric data is obtained responsive to a file import of a file associated with the ultrasound machine software;
b. determining virtual reality information representing a virtual environment, wherein at least part of the said environment is based on said volumetric data, comprising:
i. receiving an input containing information of a location and rotation of a head of the user in the real-world physical space;
ii. receiving an input containing information of a location and orientation of one or more hands of the user in the real-world physical space;
iii. calculating at least one of the following: new position, scale and/or orientation of the representation of the fetal scan in the virtual reality environment, wherein the new position, scale and orientation is responsive to received input of the location and rotation of the head of the user and the received input of the location and the orientation of one or more hands of the user; and
iv. rendering the representation of one or more fetal scans for each eye of the user through volume rendering methods applied to the said volumetric data, in the calculated position and orientation;
c. displaying the determined virtual reality information using a near-eye display system for providing the interactive virtual reality experience.

2. The method described in claim 1, wherein the said virtual reality experience is layered over a real surrounding environment, thus forming an augmented reality experience.

3. The method described in claim 1, wherein determining virtual reality information further comprises of displaying in the virtual reality environment generated visual representations corresponding to the actual position and orientation of at least one hand of the user based on the received input of the location and rotation of the head of the user and the received input of the location and the orientation of one or more hands of the user.

4. The method described in claim 1, wherein the said virtual reality environment further comprises of a visual representation of at least one point of reference.

5. The method described in claim 1, further comprising of filtering out the structural noise present in the said volumetric data of fetal ultrasound scan using at least one or more of filtering methods, prior to determining virtual reality information.

6. The method described in claim 1, where said determining virtual reality information is further comprising of adjusting the visual parameters of said one or more fetal representations, said parameters selected from a group of opacity, color and brightness, wherein the new values are calculated from the orientation of the fetal representation in relation to the position and rotation of the head and/or one or more hands of the user.

7. The method described in claim 1, further comprising of adjusting the parameters of volume rendering method of one or more fetal representations by a user prior to determining virtual reality information for the first time.

8. The method described in claim 1, further comprising of removing at least part of the volumetric data of the fetus ultrasound, that is deemed not relevant by a user prior to determining virtual reality information for the first time.

9. The method described in claim 1, wherein calculating the new position of the representation of the fetus in the virtual reality environment comprises of:
a. calculating at least one metric of one or more vectors determined by the position of the representation of the fetus in the virtual reality environment and the positions of one or more hands; and
b. moving the representation of the fetus with a velocity and/or acceleration based on the measured metrics, thus obtaining a new position of the representation of the fetus.

10. The method described in claim 1, wherein calculating new rotation and scale of the representation of the fetus in the virtual reality environment comprises of:
c. calculating at least one metric of a vector between the position of hands of the user; and
d. rotating and scaling the representation of the fetus with a velocity and/or acceleration based on the measured metrics, thus obtaining new rotation and scale of the representation of the fetus.

11. The method described in claim 1, further comprising enabling the user to interact with virtual reality environment by allowing the user to toggle between different representations of the fetus.

12. The method described in claim 1, further comprising providing the virtual reality information to one or more additional near-eye display devices associated with at least one additional user, such that at least two users are enabled to interact with the virtual reality environment via their respective near-eye display devices substantially simultaneously.

13. A system implementing the method of claim 1 that comprises:
a near-eye display configured to project a synthetic virtual scene, into both eyes of a user, so as to provide a virtual reality view to the user;
means for determining position of hands;
a memory storing executable machine-readable instructions; and
computational hardware containing one or more physical processors configured by machine readable instructions capable of performing method.

14. The system described in claim 13, wherein the said physical processors in computational hardware further include at least one Central Processing Unit (CPU) core and at least one graphical processing unit (GPU) core, the computational hardware being configured to distribute a workload of at least displaying positioning and orienting fetal scan in the prepared virtual reality scene between the CPU and the GPU.

15. The system described in claim 13, wherein the system further includes an ultrasound imaging system operable to generate data representing a body with an ultrasound transducer.

16. The system described in claim 13, wherein the system further includes a server enabled to store ultrasound scan data and/or perform some of the workload of the method.

17. The system described in claim 13 wherein the near-eye display device is detachably attached to the processing hardware.

18. The system described in claim 13, wherein the said processing hardware in the said system is at least a part of industry-standard ultrasound imaging hardware.

19. The system described in claim 13, wherein the said file associated with the ultrasound machine software is obtained over a network.

20. The system described in claim 13, wherein one or more physical processors are further configured by machine-readable instructions to enable the user to share the virtual reality information with another user through a network.

21. The system described in claim 13, wherein generating virtual reality information further includes generation of sounds for the user, being used to create music, sound effects and commentary for the virtual reality experience.

22. The system described in claim 13 further comprising of at least one depth, infrared (IR) camera sensor and at least one IR light sources attached to the near-eye display system and an image recognition software, wherein said IR light sources project IR light on the hands of the targeted recipient, IR camera sensors register an image of target recipient hands and the image recognition software is able to provide position and orientation of the hand(s).

23. The system described in claim 13 further comprising of one or two hand-held controllers tracked by an external single or multiple positioning devices, able to provide position and orientation of one or more hands.

24. The system described in claim 13 wherein the said ultrasound machine hardware is industry-standard hardware.

25. A non-transitory computer readable medium containing program instructions for causing a computer to perform, the method.
